(19) United States
(12) Patent Application Publication
(10) Pub. No.: US 2006/0129109 A1
(43) Pub. Date: Jun. 15, 2006

(54) Reconnectable disconnect device for fluid transfer line

(76) Inventors: Scott Randall Shaw, Brentwood, TN (US); Mark Raymond Shaw, Brentwood, TN (US)

Correspondence Address: MICHAEL O. SCHEINBERG, P.O. BOX 164140, AUSTIN, TX, 78716-4140, US

(21) Appl. No.: 11/249,964

(22) Filed: Oct. 13, 2005

Related U.S. Application Data
(63) Continuation-in-part of application No. 10/694,680, filed on Oct. 28, 2003.
(60) Provisional application No. 60/618,890, filed on Oct. 14, 2004.

(51) Int. Cl. A61M 5/00

(52) U.S. Cl. 604/246

(57) ABSTRACT

An improved disconnect device suitable for use with an IV tube or other medical tubing device which can be either manually disconnected or automatically disconnected by the application of an axial force sufficiently low to prevent patient injury; which can be sterilely reattached after disconnection; which allows fluid flow in either direction; which shuts off fluid flow from both directions when disconnected; and which can be simply and inexpensively manufactured and assembled with techniques common to the injection molding, and medical products manufacturing industry.

This application is a non-provisional application of U.S. Provisional Application No. 60/618,890 entitled “Reconnectable Disconnect Device for Fluid Delivery Line” which was filed on Oct. 14, 2004, and a continuation-in-part of U.S. patent application Ser. No. 10/694,680, filed Oct. 28, 2003.

### TECHNICAL FIELD OF THE INVENTION

The present invention relates to the field of fluid transfer systems and, in particular, to an assembly for disconnecting and reconnecting fluid flow through a fluid transfer system.

### BACKGROUND AND SUMMARY OF THE INVENTION

Various types of fluid-conducting tubes are commonly used for directing fluid into or withdrawing fluids from a patient. These types of devices, collectively referred to herein as medical tubing devices, can be used, for example, to deliver medications, to withdraw fluids such as blood, or to monitor various parameters of a patient's vascular system. One such device, referred to as an intravascular (IV) administration device, allows a medical practitioner to introduce therapeutic agents, medications, nutrients, and various other fluids directly into the blood stream of a patient.

A typical prior art IV administration device is shown in FIG. 1. Such a device typically consists of an over the needle catheter 10 which is inserted into a vein or artery, usually in the patient's arm 11. The needle (not shown) is then removed and catheter 10 inserted completely into the blood vessel. The healthcare provider commonly uses surgical tape 12 to maintain the position of the catheter on the skin of the patient. One end of a flexible tube called an IV line 14 is then attached to the catheter and the other end is attached to a fluid reservoir 16, allowing fluid to flow directly from the reservoir into the patient's bloodstream. The fluid typically either drains from a reservoir positioned above the patient to feed under gravity or is delivered via an infusion pump.

In some uses, IV catheters can be utilized for a relatively short duration, for example, hours or for a few days. In other cases, IV catheters may be utilized for much longer durations, weeks or even months. Once these types of medical tubing lines are in place, it is difficult to remove and replace them. For example, removing and replacing an IV line typically requires another needle stick. This will subject the patient to increased pain. Also, certain patients have inadequate veins or compromised health conditions, which may make an additional stick difficult. Not only does this increase the discomfort to the patient, chances of accidental medical personnel sticks and exposure to blood borne pathogens are also increased.

Certain IV administration devices, such as a central line or peripherally inserted central catheter (PICC) require a surgical procedure to insert the catheter into the patient. A central line is a thin flexible silicone tube or catheter, the tip of which is placed in one of the large veins deep in the chest, such as the superior vena cava. The central line is put in under either a local or a general anesthetic. The outside end of the line is on the chest, just above one or other nipple. It then tracks under the skin for a little way before going into a large vein just behind the collarbone. From there it goes into the superior vena cava. With a PICC line, a more recent development, a catheter is inserted into one of the large veins in the arm (usually near the bend of the elbow) and from there it is threaded into the superior vena cava. Once the PICC line is in place, it will usually be taped firmly to a patient's skin with a special transparent dressing to stop the catheter from moving around or coming out of the vein. In the case of either a central line or PICC line, if the line is unintentionally pulled, the patient may have to undergo additional surgery or radiological procedures in order to re-insert the device.

Whatever the type of medical tubing device used, additional difficulties arise for patients that are ambulatory or for confused or pediatric patients. Ambulatory patients typically have to contend with medical tubing lines and fluid reservoirs when moving from one location to another. Active patients sometimes inadvertently catch tubing lines on an object, while confused or pediatric patients can pull on tubing in an attempt to remove a device. Health care workers or visitors can sometimes trip or become tangled in tubing while caring for the patient, causing injury to both the patient and the person entangled.

When these types of forces are applied to a medical tubing device, such as an IV line, the tubing itself typically does not break. Instead, the force is transferred along the tubing to the insertion point into the patient's body. In the case of a typical IV device, this means that a pull on the tubing can, in turn, pull on the IV catheter resulting in significant pain to the patient. In some cases, the catheter can actually be pulled out of the patient, interrupting the flow of medication or other fluid and necessitating another needle stick to reinsert a catheter. For medical tubing devices such as drainage catheters, central line, or PICC line, the patient might have to undergo additional surgical or radiological procedures. These additional procedures add to patient discomfort, increase medical costs, and expose patients to additional risk of infection.

The typical medical tubing device is unified and not designed to provide easy disconnection or automatic fluid flow interruption in the event of disconnection. A number of disconnect devices which interrupt fluid flow by way of a valve or other device have been described in the prior art. All of the valved disconnect devices described to date, however, suffer from design characteristics that limit their usefulness for medical tubing devices. Hence, there is a need for an improved technique to allow an IV or other medical tubing device to be either manually disconnected or to automatically disconnect at a force sufficiently low to prevent patient injury and then to be sterilely reattached without replacing the device and without subjecting the patient to another needle stick or medical procedure.

Valved assemblies for use in medical tubing devices are known in the prior art. Such devices are described, for example, in U.S. Pat. No. 6,036,171 to Weinheimer et al. for “Swabbable Valve Assembly,” in U.S. Pat. No. 5,700,248 to Lopez for “Medical Valve with Tire Seal,” and in U.S. Pat. No. 5,137,524 to Lynn et al. for “Universal Intravenous Connector with Dual Catches.” Many such devices connect by way of a needle piercing a septum. Thus, such devices generally only allow fluid flow in one direction. Also, repeated piercing of the needle through the septum can damage the septum, resulting in leaks or in the introduction of material from the septum into the flow line. Further, even where a needle and septum arrangement is not employed, these types of devices typically only shut off fluid flow in one direction when disconnected. Finally, none of these designs allows for automatic disconnection if a predetermined force is applied to the tubing.

A valved assembly that does allow for automatic disconnection is described in U.S. Pat. No. 5,820,614, to Erskine et al. for “Disconnect for Medical Access Devices.” However, this device, due to a number of design limitations, does not adequately address many of the common problems with the use of IV lines that are encountered in the modern medical facility.

For example, the Erskine design does not adequately provide for intentional manual disconnection. The only method for intentionally disconnecting the device is to apply a distal axial load to the connector or tubing, thus pulling the connector apart. As a result, each disconnect—whether intentional or automatic—will cause additional wear on the collar and shoulder latching assembly, which will decrease the number of additional times that the device may be disconnected and reconnected before the latching assembly is worn out. The use of a spring in the design increases the cost of the device due to the cost of the spring and the increased assembly time. The presence of the spring and the chamber housing the spring also increases the possibility of bacterial contamination. In the event that fluid seeps into the spring housing, a stagnant fluid pool could be created allowing bacteria to reproduce. Because the septa are located behind (distal to) the connecting mechanism, the Erskine design does not permit easy access to critical sites that must be disinfected by swabbing with alcohol or other appropriate disinfectant solution. Further, the design of the piercing cannula subjects the septum to abrasive forces which degrade the material and lead to significant generation of debris which may be transported into the patient.

Although there are also numerous break-away hose connectors known and used in other fields, such as the gasoline-dispensing device described in U.S. Pat. No. 4,905,733, these devices do not permit easy disinfection, which is critical in medical devices. In the above cited patent, a ball mechanism is mounted within the latch. This mechanism is exposed to the fluid. The non-smooth surface prevents easy “swabbability” or disinfection of the device, thereby rendering the design unsuitable for the medical field.

Hence, there is a need for an improved technique to allow an IV or other medical tubing device to be either manually disconnected or to automatically disconnect at a force sufficiently low to prevent patient injury and then to be sterilely reattached without necessitating another needle stick or medical procedure to replace the device; which allows fluid flow in either direction; which shuts off fluid flow from both directions when disconnected; and which can be simply and inexpensively manufactured and assembled with techniques common to the injection molding, and medical products manufacturing industry. The disconnection device described below derives new and unique benefits from a combination of valving and latching elements not revealed before.

### SUMMARY OF THE INVENTION

An object of the invention, therefore, is to provide an improved disconnect device suitable for use with an IV tube or other medical tubing device which can be either manually disconnected or automatically disconnected by the application of an axial force sufficiently low to prevent patient injury. Another object of the invention is to provide an improved disconnect device which can be easily disinfected and reconnected without subjecting the patient to another needle stick or medical procedure or the need to replace the medical tubing device.

The foregoing has outlined rather broadly the features and technical advantages of the present invention in order that the detailed description of the invention that follows may be better understood. Additional features and advantages of the invention will be described hereinafter. It should be appreciated by those skilled in the art that the conception and specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 shows a typical prior art IV administration device.

FIG. 2 is a cross-sectional view of a disconnect device according to the present invention with the male and female connectors disengaged.

FIG. 3A is a cross-sectional view of the penetration tube of the disconnect device of FIG. 2.

FIG. 3B is a perspective view of the penetration tube of the disconnect device of FIG. 2.

FIG. 3C is another perspective view of the penetration tube of the disconnect device of FIG. 2.

FIG. 4A is a cross-sectional view of the male and female stopples of the disconnect device of FIG. 2.

FIG. 4B is a cross-sectional view of the assembled male stopple and penetration tube of the disconnect device of FIG. 2.

FIG. 5 is a cross-sectional view of the disconnect device of FIG. 2 with the male and female connectors fully engaged.

FIG. 6A is a cross-sectional view of the male connector of FIG. 2 showing the manual disconnect feature.

FIG. 6B is a different cross-sectional view of the male connector of FIG. 2.

FIG. 6C is a perspective view of the male connector of FIG. 2.

FIG. 7A is a perspective view of one embodiment of a disconnect device according to the present invention with the male and female connectors disengaged but oriented for proper attachment.

FIG. 7B is a perspective view of one embodiment of a disconnect device according to the present invention with the male and female connectors fully engaged.

FIG. 8 is a perspective view of one embodiment of a disconnect device according to the present invention showing a different pattern of alignment wings and grooves.

FIG. 9 is a perspective view of a disconnect device according to the present invention where the male connector can be attached directly to a prior art needleless access port by using an adapter ring.

FIG. 10 is a perspective view of the disconnect device of 9 with male connector and needleless access port (with attached adapter ring fully engaged.

FIG. 11A is a cross-sectional view of the adapter ring of the disconnect device of FIG. 9 and FIG. 10.

FIG. 11B is a perspective view of the adapter ring of the disconnect device of FIG. 9 and FIG. 10 as seen from a side angle.

FIG. 12A is a cross-sectional view of the male connector and adapter ring (attached to a prior art needleless access port) of the disconnect device of FIG. 9 and FIG. 10.

FIG. 12B is a different cross-sectional view of the male connector of the disconnect device of FIG. 9 and FIG. 10.

FIG. 13A is a cross-sectional view of the male stopple of the disconnect device of FIG. 9 and FIG. 10.

FIG. 13B is a perspective view of the male stopple of the disconnect device of FIG. 9 and FIG. 10 as seen from a side angle.

FIG. 14A is a cross-sectional view of the penetration tube of the disconnect device of FIG. 9 and FIG. 10.

FIG. 14B is a perspective view of the penetration tube of the disconnect device of FIG. 9 and FIG. 10 as seen from a side angle.

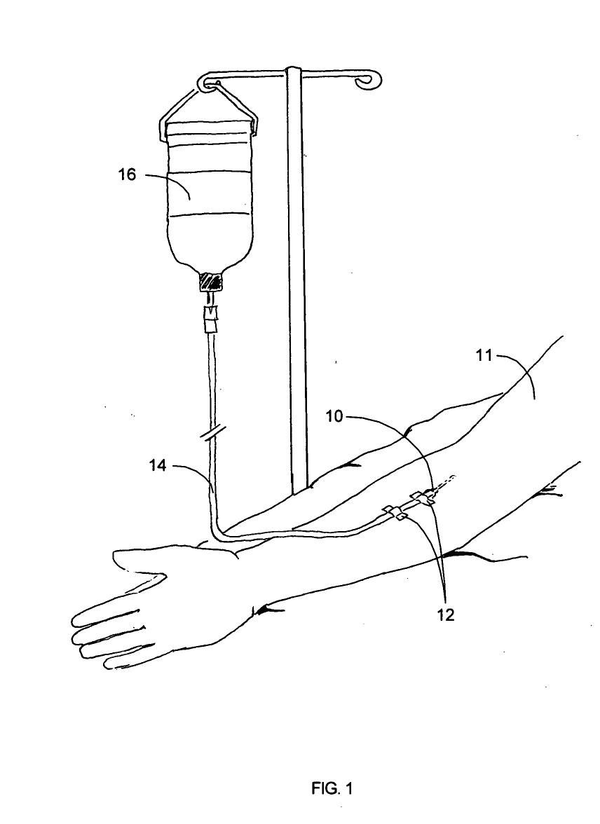

### DETAILED DESCRIPTION OF PREFERRED EMBODIMENTS

A preferred embodiment of this invention provides a novel apparatus allowing an IV tube or other medical tubing device to be either manually disconnected or to automatically disconnect at a force sufficiently low to prevent patient injury and then to be reattached without necessitating another needle stick or medical procedure or the need to replace the device.

In accordance with one aspect of a preferred embodiment of the present invention, the design of the apparatus allows fluid flow across a fluid delivery tubing device to be quickly disconnected without significant leakage of fluid.

In accordance with another aspect of a preferred embodiment of the present invention, the design of the apparatus allows fluid flow across a fluid delivery tubing device to be automatically disconnected if a force above a certain threshold is applied to the apparatus itself or to the fluid delivery line.

In accordance with another aspect of a preferred embodiment of the present invention, the design of the apparatus prevents any significant fluid leakage and prevents the introduction of foreign substances or contaminants into the fluid supply when fluid flow is disconnected.

In accordance with another aspect of a preferred embodiment of the present invention, the design of the apparatus allows disconnected fluid flow across a fluid delivery tubing device to be reconnected after appropriate disinfection of connecting surfaces.

In accordance with another aspect of a preferred embodiment of the present invention, the design of the apparatus allows different sets of connectors with distinct geometric configurations to provide for a means of preventing improper connections in a multiple connection environment.

In accordance with another aspect of a preferred embodiment of the present invention, the design of the apparatus allows for an alarm when fluid flow is disconnected.

Particular embodiments of the present invention are directed to an apparatus for connecting and disconnecting opposite ends of a liquid flow line. Although much of the following description is directed toward medical tubing devices, the apparatus could equally be utilized with any type of liquid flow device. Hence, the scope of the present invention should not be limited to a disconnect device for medical tubing devices. In this application, the terms “valve,” “septum,” and “stopple” will be used interchangeably to refer to devices for opening, closing, or modifying the flow of a fluid through a tube, outlet, inlet, or the like. Further, in this application, the term “proximal” will be used to designate the end of the connector nearest the opposing connector and the term “distal” will be used to designate the end of the connector furthest from the opposing connector. The term “distal axial force” will be used to describe force applied along the longitudinal axis of the connector, parallel to the fluid flow, in a direction that pulls the two opposing connectors apart. It will be assumed for the sake of simplicity that fluid flow occurs from the male connector to the female connector. However, it will be apparent to those skilled in the art that when the two connectors are engaged fluid can flow in either direction and from either connector.

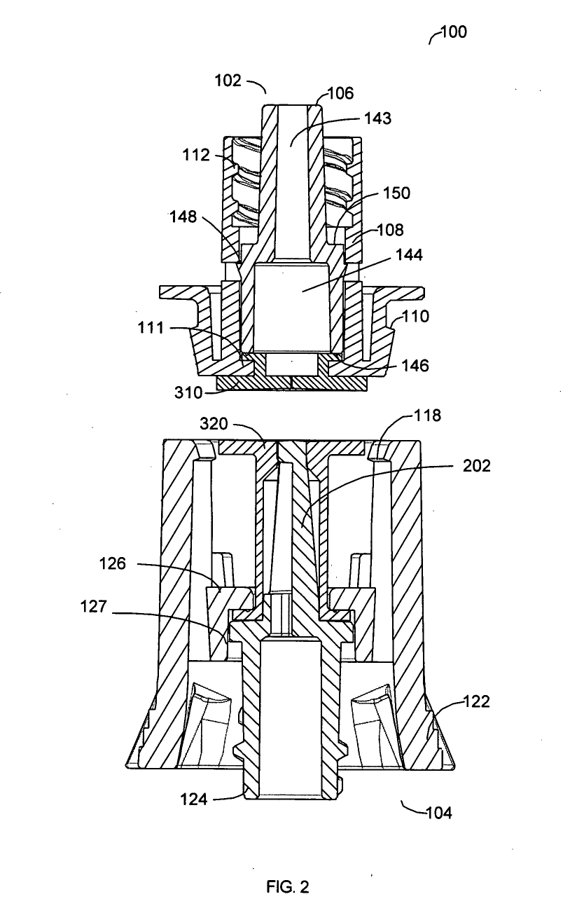

FIG. 2 is a cross-sectional view of a disconnect device according to the present invention. In a preferred embodiment, the disconnect device comprises two connectors, a female connector 102 and a male connector 104. Each connector can be attached to corresponding sections of a medical tubing line (not shown). For example, female connector 102 could be connected to one end of a length of IV tubing and the other end connected to the catheter inserted into a patient's bloodstream; and male connector 104 could be connected to one end of a second length of IV tubing with the other end of the second length connected to a fluid reservoir containing therapeutic agents, medications, nutrients, or various other fluids. Alternatively, either the female connector 102 or the male connector 104 could be connected directly to the catheter inserted into a patient's body.

In another preferred embodiment, either the female connector 102 or the male connector 104 could be either permanently bonded to the catheter inserted into the patient or manufactured as a part of said catheter. Alternatively, the other connector could be permanently bonded to the supply or drainage tubing. In this embodiment, the luer lock connectors described below could be eliminated resulting in a significant reduction in the outer diameter of the connectors.

In the disengaged view of FIG. 2, the male and female connectors are disposed generally opposite each other. Female connector 102 comprises a female connector housing 108, which contains luer body 106 and female connector stopple 310. Female connector housing 108 is generally cylindrical in shape and includes stopple support 111, external female connector detents 110, and internal luer lock threads 112.

Male connector 104 comprises a male connector housing 122, which contains penetration tube 202 and male connector stopple 320. Male connector housing 122 is also generally cylindrical in shape and includes male connector detents 118 and internal sealing support 126.

Preferably, male connector housing 122 is formed from a material that has a high degree of flexibility and lubricosity such as high-density polyethylene. Female connector housing 108, luer body 106, and penetration tube 202 can be formed from a thermoplastic material such as high-impact polystyrene. Female connector stopple 310 and male connector stopple 320 can be formed from a resilient elastomeric material such as rubber or a silicon elastomer.

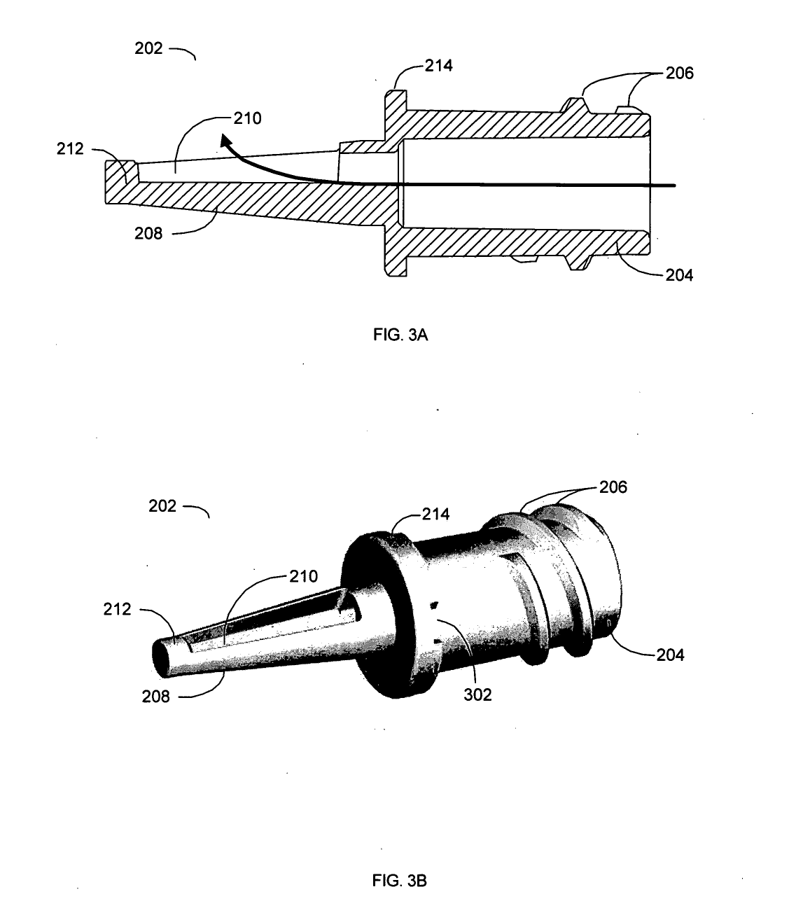

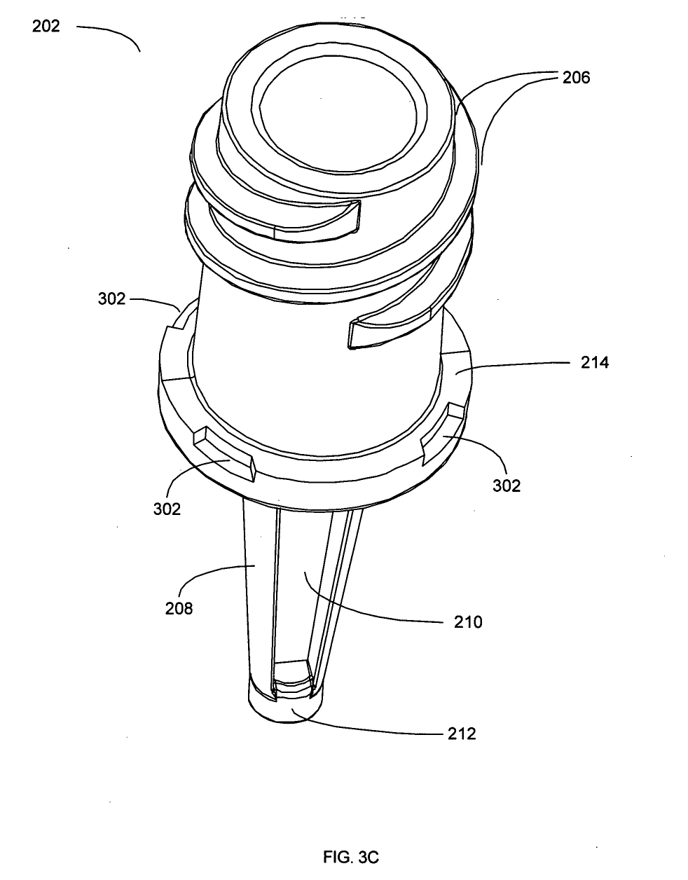

FIG. 3A is a cross-sectional view of the penetration tube 202 of the disconnect device of FIG. 2. FIG. 3B is a perspective view of the penetration tube of the disconnect device of FIG. 2 as seen from the side. FIG. 3C is a perspective view of the penetration tube of the disconnect device of FIG. 2 as seen from the distal end. Penetration tube 202 preferably has a generally cylindrical base 204, which can be threaded with external luer threads 206, and a generally conical tip 208 with fluid transfer opening 210 and cap 212. Optionally, cylindrical base 204 can be hermetically attached to tubing eliminating the need for luer threads 206. At its apex, conical tip 208 is topped by cap 212, which preferably has a smooth or blunt proximal surface. External luer threads 206 on penetration tube base 204 can be used to connect a fluid transfer line such as a conventional IV line. The interior of base 204 and tip 208 are continuous such that liquid flowing into base 204, for example fluid flowing through an IV line, passes into tip 208 and exits through one or more fluid transfer openings 210.

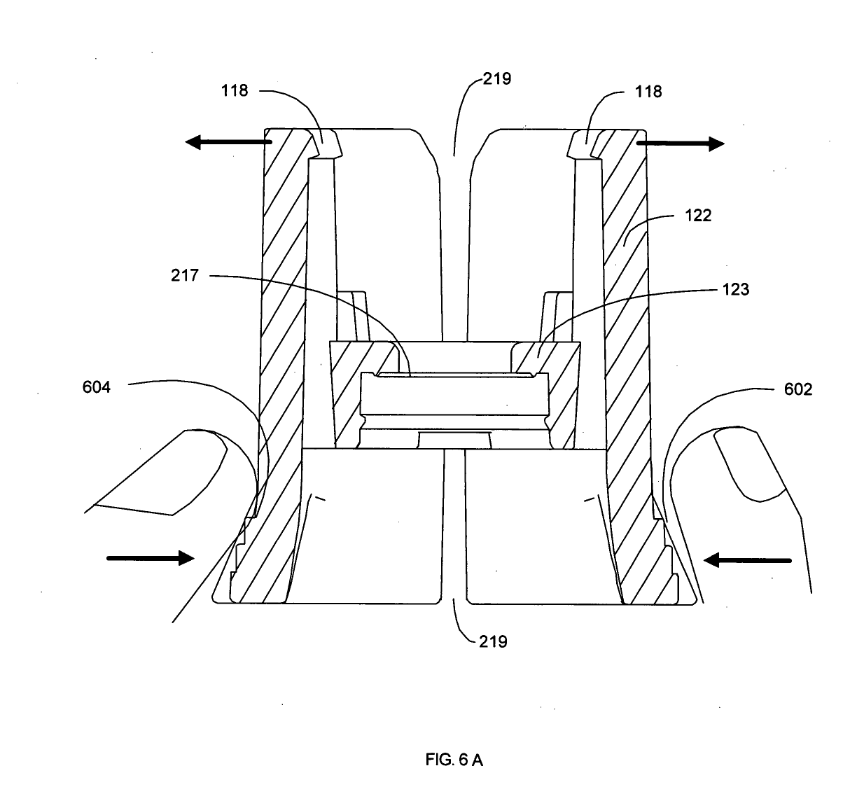

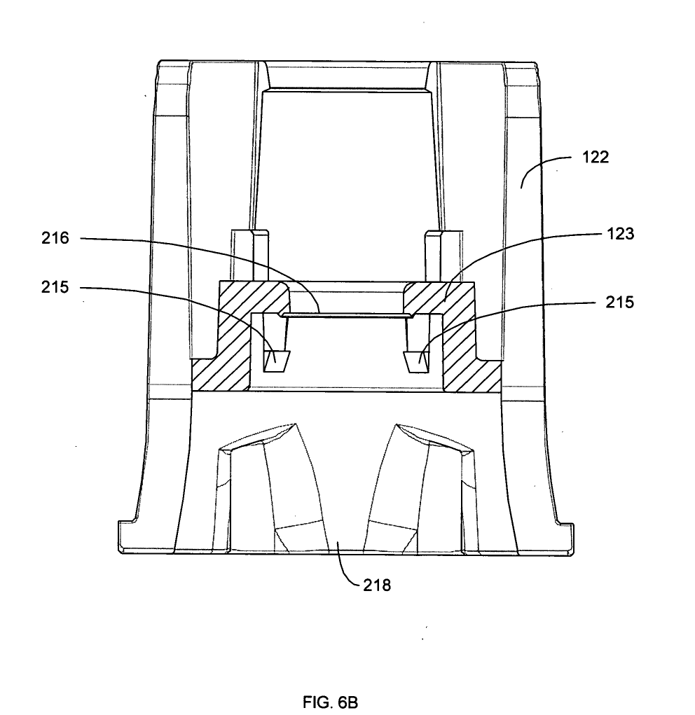

Referring also to FIG. 6A and FIG. 6B, penetration tube 202 can be held in place in male connector housing 122 by tube retainer latches 215. In a preferred embodiment, when penetration tube 202 is mounted into male connector housing 122, tube retaining latches 215 compress as tube retainer ring 214 passes, then expand to lock into latch grooves 302 located on the distal surface of tube retainer ring 214, thereby holding tube 202 in its final position. Alternatively, tube 202 can be held in connector housing 122 with adhesive bonding or sonic welding. Retaining collar 322, the flange portion at the distal end of stopple 320 as discussed below, is compressed between the proximal surface of tube retainer ring 214 and a sealing bead 217 on the interior surface of internal sealing support 126. Preferably, the connection between penetration tube 202 and male connector housing 122 is capable of withstanding an axial load of 25N in accordance with International Standard ISO 594-1 (1986 ed., Reference No. ISO 594/1-1986(E)) as published by the International Organization for Standardization, Case postale 56, CH-1211 Geneva 20, Switzerland.

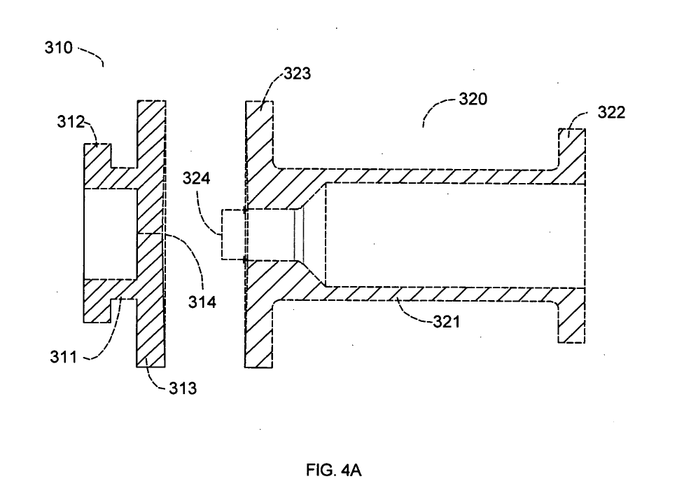

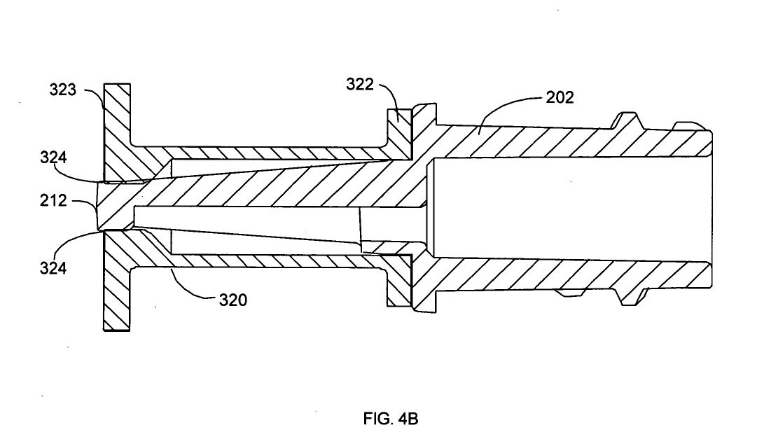

FIG. 4A is a cross-sectional view of the male and female stopples of the disconnect device of FIG. 2. Both the male and female stopples comprise valves that are normally biased so as to block any fluid flow through either connector when the male and female connectors are disengaged. Male connector stopple 320 is generally spool shaped with a cylindrical center section 321 and flange portions formed at either end of the cylindrical section. The flange portion at the distal end of stopple 320 forms retaining collar 322. The flange portion at the proximal end of stopple 320 forms male septum 323, with formed hole 324 located in the center of male septum 323.

Male connector stopple 320 can be mounted in male connector housing 122 by compressing retaining collar 322 and pulling the compressed ring through the center opening in internal sealing support 126 and toward the distal end of male connector housing 122. As shown in FIG. 4B, penetration tube 202 is seated inside stopple 320 oriented so that tip of penetration tube 202 is proximal to its base. In a preferred embodiment, cap 212 passes through formed hole 324 and protrudes slightly from the proximal surface of male septum 323. This arrangement provides for a tight seal while at the same time minimizing damage to the septum that could result from repeated piercing of the septum by the tip of the penetration tube 202. The proximal sealing surface of penetration tube 202 compresses retaining collar 322 against the distal surface of internal sealing support 126. The connection at the joint between the penetration tube 202, male stopple retaining collar 322, and the distal surface of internal sealing support 126 should preferably be capable of sealing water pressure at 300 kPa in accordance with Part 5.2 of the International Standard ISO 594-2 (1998 ed., Reference No. ISO 594-2: 1998(E)) as published by the International Organization for Standardization, Case postale 56, CH-1211 Geneva 20, Switzerland.

Female connector stopple 310 is also generally spool shaped with a cylindrical center section 311 and flange portions formed at either end of the cylindrical section. The flange portion at the distal end of stopple 310 forms sealing ring 312. The flange portion at the proximal end of stopple 310 forms female septum 313 with slit 314. Referring also to FIG. 2, female connector stopple 310 is held in place by female stopple support 111. Stopple 310 can be mounted, for example, by compressing sealing ring 312 and pushing it through the center of stopple support 111. Stopple support 111 thus fits inside a retaining groove formed by the cylindrical and flange portions of stopple 310.

Referring again to FIG. 2, luer body 106 is inserted into the distal end of female connector housing 108 and oriented so that fluid chamber 144 is proximal to luer taper 143. Luer body 106 can be held in place, for example, by compressible luer retaining latches 148 formed on the exterior surface of luer body 106 just proximal to luer body shoulder 150. In a preferred embodiment, as luer body 106 is inserted, luer retaining latches 148 press against the interior surface of female connector housing 108. Luer retaining latches 148 are compressed as luer body 106 is pressed into female connector housing 108 and then expand into mounting cavities 149 to hold luer body 106 in its final position. Once luer body 106 is fully inserted into female connector housing 108, luer sealing ring 146 at the proximal base of fluid chamber 144 presses against the distal surface of female stopple sealing ring 312, thus compressing sealing ring 312 against the distal surface of female stopple support 111 and forming a tight fluid seal.

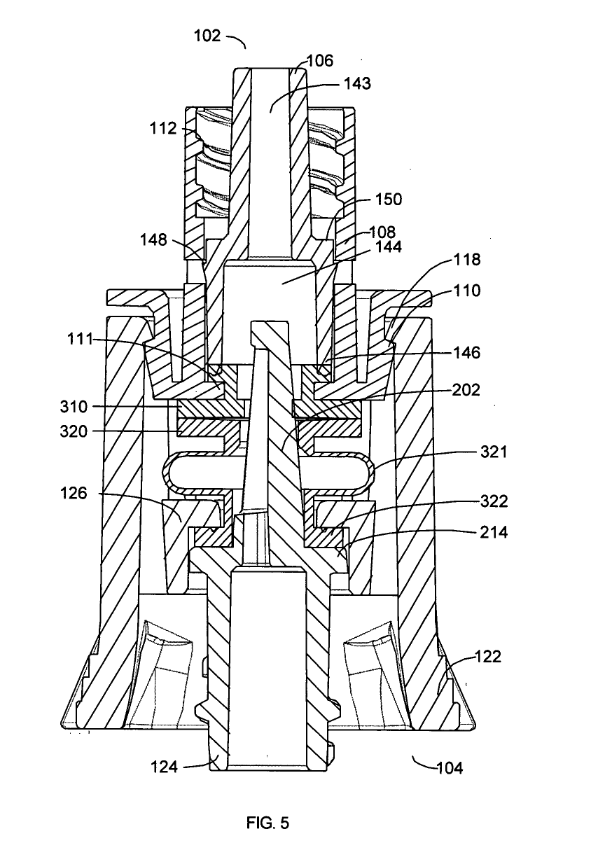

FIG. 5 is a cross-sectional view of the disconnect device of FIG. 2 with the male and female connectors fully engaged. When disengaged, the stopples of both connectors are biased so as to block any fluid flow through either connector. In order to engage the connectors and overcome the stopples' normal bias thereby allowing fluid flow through the connectors, the male and female connectors are first brought into approximate center alignment so that the proximal surface of female connector septum 313 is in contact with the proximal surface of male connector septum 323. As discussed in greater detail below, alignment features in the connectors can be used to ensure proper orientation. As shown in FIG. 4A, both female septum 313 and male septum 323 have slightly concave surfaces. As a result, when the septa are brought together the concave edges will compress thus creating a slight suction force sealing the surfaces of the septa together.

As the connectors are pushed together, female connector stopple 310 is held in place by female stopple support 111. The cylindrical center section 321 of male connector stopple 320 is formed with collapsible sidewalls, which allow male connector stopple 320 to compress axially towards the distal end of the tip 208 of penetration tube 202. Formed hole 324 in male septum 323 expands radially to accommodate and seal around the increasing diameter of tip 208 as male connector stopple 320 is compressed. At the same time, tip 208 pierces the slit 314 in female septum 313 and inserts into fluid chamber 144 of luer body 142. Preferably, penetration tube 202 has a smooth or blunt end which minimizes coring damage to the septum that could result from repeated piercing of the septum by a penetration member with sharper edges such as the tube or cylinder designs taught by the prior art. Slit 314 in female septum 313 also expands radially to accommodate and seal around the increasing diameter of tip 208 as the tip is inserted. When male connector stopple 320 is completely compressed, fluid transfer opening 210 extends at least partially into fluid chamber 144, thus allowing fluid to flow from a fluid source (not shown) through penetration tube 202, into fluid chamber 144, and then out into the downstream portion of the fluid-delivery device (not shown). In a preferred embodiment, formed hole 324 displaces radially when tip 208 extends through it, and therefore does not deform the male septum 323 outward into the female septum 313, which helps maintain the fluid tight seal between the two septa and also lowers the required attachment force.

As discussed in greater detail below, once the connectors are brought together in the fully engaged position, female connector detents 110 and male connector detents 118 engage to hold the connectors in place and maintain fluid connection. Although the detents described in this embodiment are essentially latches with lips and opposing faces that catch to hold the detents together, skilled persons will recognize that a number of different types of detents could be used, including but not limited to additional latching mechanisms such as magnetic, adhesive, or hook and loop connections. In a preferred embodiment, engagement of the female connector detents 110 and male connector detents 118 will result in an audible “click” indicating the proper engagement of the two components. In this locked position, fluid can flow from penetration tube base 204 to penetration tube tip 208, out through fluid transfer opening 210, into fluid chamber 144 of luer body 142, and then into a fluid delivery tube attached to luer body 142.

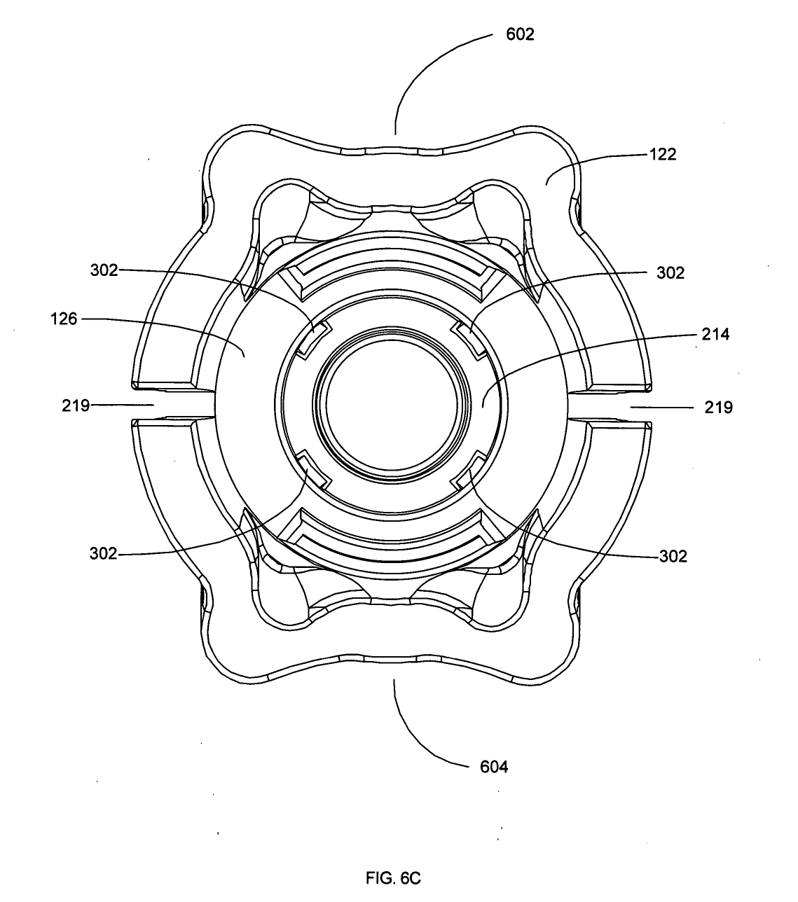

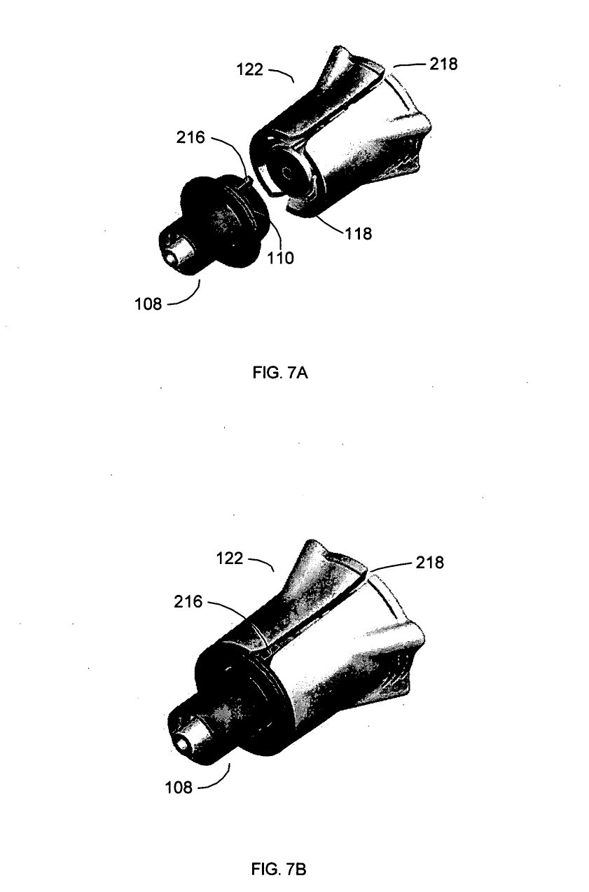

FIG. 6A is a cross-sectional view of the male connector of FIG. 2 showing an embodiment of a manual disconnect feature according to the present invention. FIG. 6B is a different cross-sectional view of the male connector of FIG. 2 at a plane 90 degrees from the cross-section shown in FIG. 6A. FIG. 6C is a perspective view of the male connector of FIG. 2. Referring also to FIGS. 7A and 7B, in this preferred embodiment, disconnect slots 218 extend the entire length of male connector housing 122, essentially dividing male connector housing 122 into two halves connected by mounting hinges 123. In a preferred embodiment, interior sealing support 126, discussed above, can also serve as such a mounting hinge. In a preferred embodiment, the application of inward force on the distal end of male connector housing 122 at points approximately 90 degrees from the disconnect slots 218 can cause the two halves of male connector housing 122 to pivot at mounting hinge 123. This application of force will narrow the width of disconnect slots 218 at the distal end of male connector housing 122 while simultaneously expanding the width of disconnect slots 218 at the proximal end of male connector housing 122. Sufficient expansion of the proximal end of disconnect slots 218 will cause female connector latch 110 and male connector latch 118 to disengage.

Such an application of force can be accomplished, for example, by squeezing together finger grip 602 and corresponding opposite finger grip 604. Once the male and female connector detents are disengaged, male connector stopple 320—which is in a compressed state when the two connectors are engaged—will expand axially towards the proximal end of the tip 208 of penetration tube 202. Formed hole 324 in male septum 323 contracts radially to retain a seal around the decreasing diameter of tip 208 as male connector stopple 320 is expanded.

The expansion of male connector stopple 320 serves to apply pressure against the proximal surface of female connector septum 313, thus pushing female connector 102 away from penetration tube 202 and removing tip 208 from fluid chamber and female septum slit 314. Slit 314 will then reseal preventing any fluid back-flow. When male connector stopple 320 is completely expanded, the perimeter of cap 212 again seals formed hole 324 in male connector septum 323 at a point proximal to fluid transfer opening 210, thus preventing any further flow through male connector 104. In order to completely disengage the connectors, the male and female connectors must be pulled apart with sufficient additional force to overcome the slight suction force resulting from the concave surface of the two septa, as discussed above.

In a preferred embodiment, the disconnect device of the present invention can also serve as a break-away device allowing automatic disconnection when sufficient distal axial force is applied to either the fluid delivery device or the disconnect device. The application of sufficient distal axial force to the fully engaged disconnect device shown in FIG. 5 can pull female connector detents 110 past male connector detents 118, thus disconnecting the two connectors and shutting off fluid flow. By varying the materials used in construction of the connector bodies, varying the amount of overlap between female connector detents and male connectors detents when the connectors are fully engaged, and varying the angle of the latch faces of female connector latch 110 and male connector latch 118, the distal axial force required to separate the male and female connectors can be varied. Selection of a disengagement force which is less than a certain threshold—for example, a threshold less than the force required to pull an IV catheter out of a patient's bloodstream or the force required to pull a drainage catheter out of the patient—would allow the disconnect device to separate before the force resulted in injury to the patient.

In a preferred embodiment of the present invention, after either manual or automatic disengagement, the disconnect device can be easily disinfected so that the device can be reconnected and the fluid flow restored without introducing any pathogens or contaminants into the fluid supply. Referring again to FIG. 2, the illustrated embodiment comprises male and female proximal septa surfaces that are easily accessible when the connectors are disengaged. The surfaces of these septa can, for example, be disinfected via the application of a conventional disinfecting solution with a swab or other means.

In a preferred embodiment of the present invention, a conventional proximity switch or contact can be placed at or near the male and female connector stopples to alert health care personnel of a disconnection by, for example, sounding an audible alarm.

FIG. 7A is a perspective view of one embodiment of a disconnect device according to the present invention with the female connector 102 and male connector 104 disengaged but oriented for proper attachment. FIG. 7A also shows female connector latch 110 and male connector latch 118 which can be engaged to hold the connectors in place. FIG. 7B is a perspective view of one embodiment of a disconnect device according to the present invention with the female connector 102 and male connector 104 fully engaged. Anti-rotation alignment wings 216 formed on female connector housing 108 can be aligned with corresponding anti-rotation grooves or slots formed in male connector housing 122. Once the female connector 102 and male connector 104 are fully engaged, the anti-rotation wings can serve to prevent the female connector 102 from rotating relative to the male connector 104. This allows the assembled connector to be gripped to tighten the male and female luer connectors in order to attach tubing to the female connector 102 and/or male connector 104. In the embodiment shown in FIG. 7A and FIG. 7B, disconnect slots 218 serve as anti-rotation grooves.

Although the illustrated embodiment has two anti-rotation alignment wings 216 located 180 degrees apart, skilled persons will recognize that different numbers and orientations of corresponding alignment wings and grooves can be utilized. Different alignment sets could be used for different types of fluid transfer lines, such as for example IV lines and drainage lines, to prevent accidental connection of the wrong fluid transfer lines in a multiple connection environment. Other systems could easily be employed to prevent accidental connection of different fluid lines, including for example different sets of connectors with distinct geometric configurations or different patterns of interlocking pins and holes on the male and female housings.

Optionally, tubing can be permanently bonded to female connector 102 and/or male connector 104, eliminating the need for any anti-rotation means. In that case, however, different alignment sets as discussed above could still be employed to prevent accidental connection of different fluid lines.

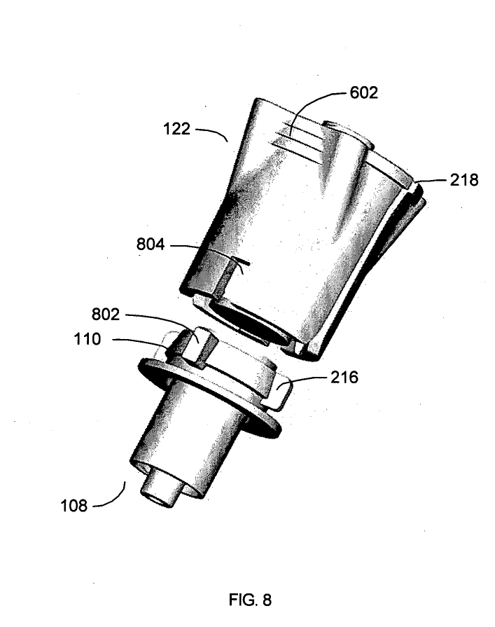

FIG. 8 is a perspective view of one embodiment of a disconnect device according to the present invention showing a different pattern of alignment wings and grooves. In order to attach the connector shown in FIG. 8, alignment wing 802 must be lined up with alignment groove 804. As shown in FIG. 7A and FIG. 7B, alignment wings 216 can be lined up with disconnect slots 218, which in this instance also serve as alignment grooves. As the male and female connectors are engaged, alignment wing 802 will slide into alignment groove 804 and alignment wings 216 will slide into disconnect slots 218. If different patters of alignment wings and grooves were employed for different fluid transfer lines, mismatched male and female connectors could not be engaged thus preventing accidental connection of the wrong fluid transfer lines in a multiple connection environment.

In another preferred embodiment, the male connector can be configured to attach directly to a conventional needleless medical connector or access port. Needleless medical connectors for injecting fluid into or removing fluid from a medical tubing device, such as an IV system, are well known and widely used. Conventional needleless medical connectors generally include a housing having an inlet port and an outlet port. The outlet port of the connector is typically connected to IV tubing, which in turn is connected to an IV catheter that communicates with a patient's venous system. The inlet port is sized to receive a blunt male cannula, such as a standard male luer tip. Disposed within the inlet port is a valve mechanism that allows any standard male luer tip to open the connection. When the male luer tip is removed, the valve will reseal. The exterior of the connector housing will typically have a threaded portion that can be securely connected to the internal threads on a standard luer lock collar. To insure universal connections between components provided by different manufacturers, universal standards for medical connectors have been developed by the International Organization for Standardization (ISO standards) and the American National Standards Institute (ANSI standards).

Configuring the male connector of the present invention to attach directly to a conventional needleless medical connector or access port provides a number of advantages. It allows the access port to be used to administer additional IV medications, while the quick disconnect/reconnect function allows the IV line to be momentarily disconnected in order to administer medication through the access port via a syringe. This embodiment also has the advantage of lower material costs since no female connector is required and the embodiment makes use of the existing access port and valve.

Skilled persons will recognize that the physical connection of the male connector of the present invention to a standard needleless access port can be accomplished in a number of different ways. For example, the connection could be accomplished by way of an adapter ring that connects to the threaded housing of a standard needleless access port. The use of such an adapter ring would allow the use of a standard male connector housing as discussed above. A needleless access port could also be manufactured with a built-in connection means, such as the latch system discussed above, thus eliminating the need for an adapter ring. Alternatively, the connection means of the male connection housing could be adapted to attach directly to the standard external threads of a conventional needleless access port.

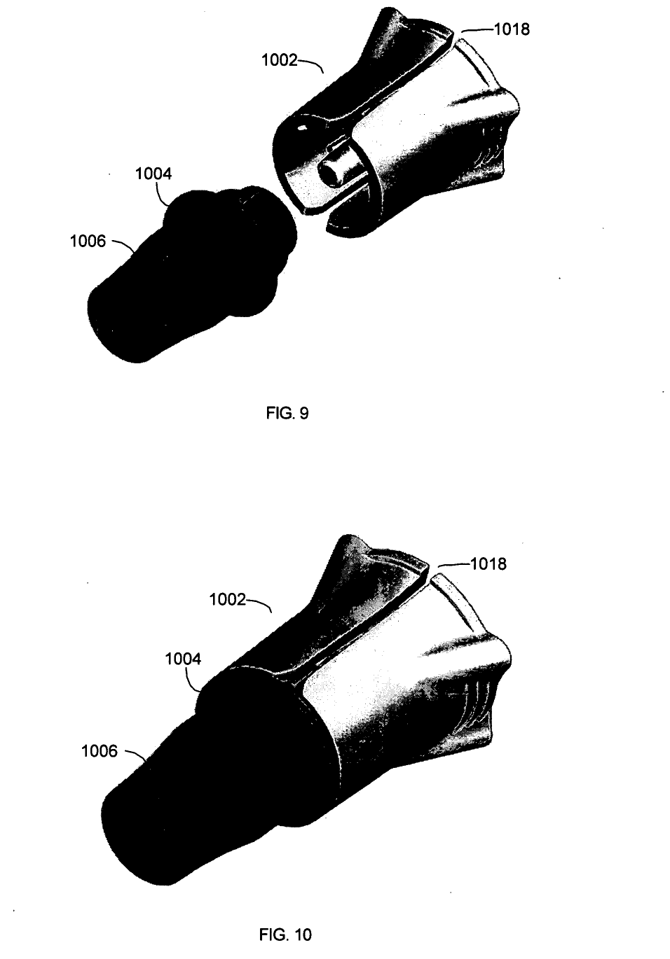

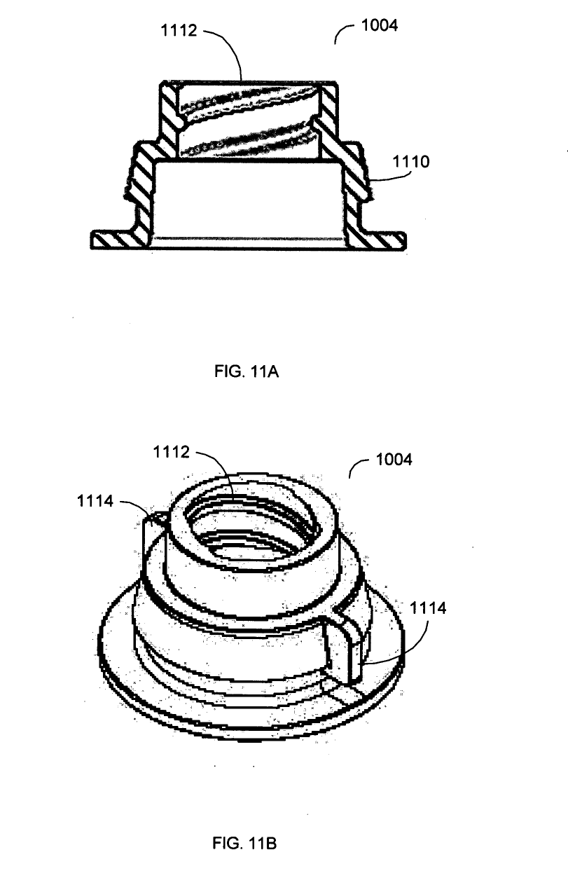

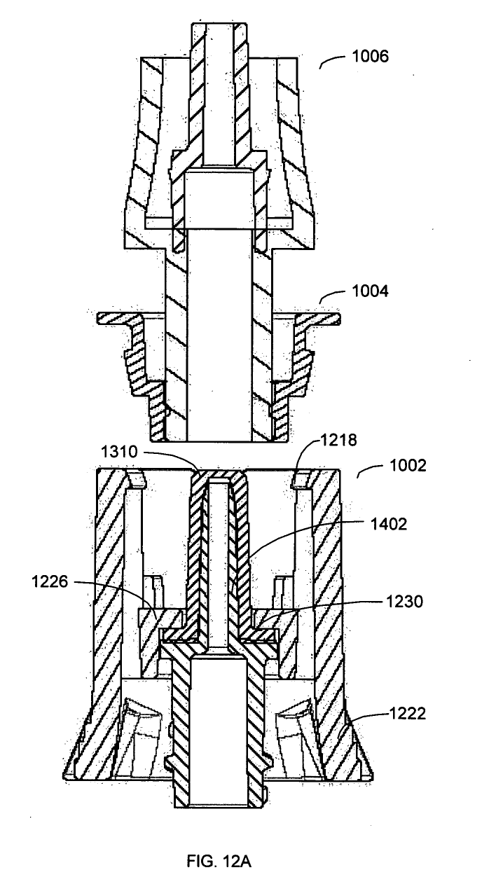

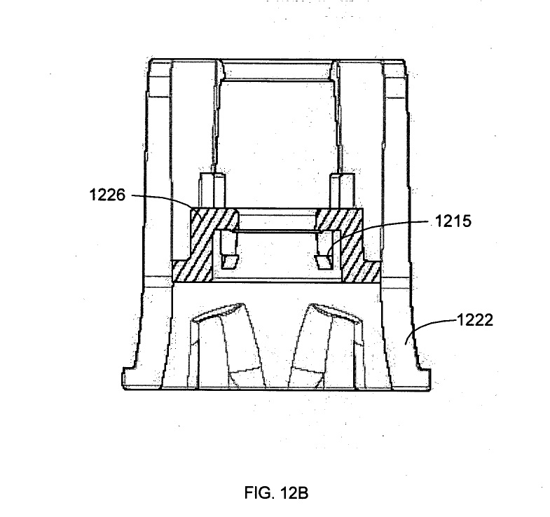

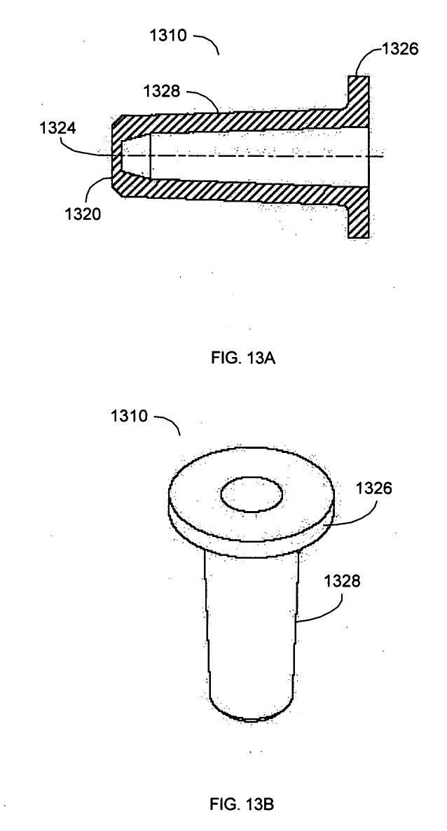

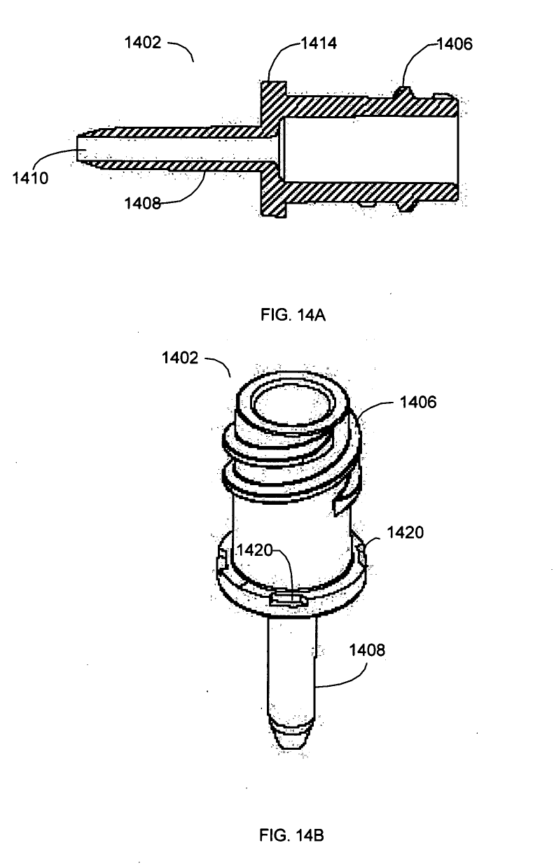

FIG. 9 and FIG. 10 are perspective views of a disconnect device according to the present invention where the male connector 1002 can be attached directly to a prior art needleless access port 1006 by using an adapter ring 1004. In FIG. 9, male connector 1002 and needleless access port 1006 (with attached adapter ring 1004) are disengaged but oriented for proper attachment. FIG. 10 is a perspective view of the disconnect device of 9A with male connector 1002 and needleless access port 1006 (with attached adapter ring 1004) fully engaged. FIG. 11A is a cross-sectional view of the adapter ring 1004 of the disconnect device of FIGS. 9A and 9B. FIG. 11B is a perspective view of the adapter ring 1004 of the disconnect device of FIGS. 9A and 9B as seen from a side angle. FIG. 12A is a cross-sectional view of the male connector 1002 and adapter ring 1004 (attached to a prior art needleless access port 1006) of the disconnect device of FIG. 9A and FIG. 9B. FIG. 12B is a different cross-sectional view of the male connector 1002 of the disconnect device of FIG. 9A and FIG. 9B. FIG. 13A is a cross-sectional view of the male stopple 1310 of the disconnect device of FIGS. 9A and 9B. FIG. 13B is a perspective view of the male stopple 1310 of the disconnect device of FIGS. 9A and 9B as seen from a side angle. FIG. 14A is a cross-sectional view of the penetration tube 1402 of the disconnect device of FIGS. 9A and 9B. FIG. 14B is a perspective view of the penetration tube 1402 of the disconnect device of FIGS. 9A and 9B as seen from the side.

In this preferred embodiment, adapter ring 1004 attaches to the standard external luer lock threads of a prior art needleless access port 1006. Adapter ring 1004 is generally cylindrical in shape and includes external adapter ring detents 1110, internal luer lock threads 1112, and anti-rotational alignment wings 1114. Although the illustrated embodiment has two anti-rotation alignment wings 1114 located 180 degrees apart, skilled persons will recognize that different numbers and orientations of corresponding alignment wings and grooves can be utilized as discussed above.

Male connector 1002 preferably comprises a male connector housing 1022, which contains penetration tube 1402 and male connector stopple 1310. Male connector housing 1222 is also generally cylindrical in shape and includes male connector detents 1218 and internal sealing support 1226. In this embodiment, penetration tube 1402, discussed in greater detail below, is modified from embodiments discussed above so that penetration tube 1402 in conjunction with stopple 1310 will substantially conform to ISO and ANSI standards for a male luer tip so that male connector 1002 can connect to a conventional needleless medical connector.

When disengaged, the stopple 1310 of male connector 1002 and the valve of the prior art needleless access port 1006 are biased so as to block any fluid flow through either the connector 1002 or the access port 1006. Male connector stopple 1310 is generally cylindrically shaped with a flange shaped retaining collar 1326 formed at the distal end of the cylindrical section. The proximal end of stopple 1310 comprises male septum 1320 with stopple slit 1324 located in the center of male septum 1320. In this embodiment, the cylindrical center section 1328 of male connector stopple 1310 is tapered to fit a penetration tube 1402, as discussed above, so that the stopple and tip together substantially conform to ISO and ANSI standards for a male luer tip.

Penetration tube 1402 is seated inside stopple 1310 oriented so that tip of penetration tube 1402 is proximal to its base. Penetration tube 1402 and male stopple 1310 are seated inside male connector housing 1222. The proximal sealing surface of penetration tube 1402 compresses retaining collar 1326 against the distal surface of internal sealing support 1226. The connection at the joint between the penetration tube 1402, male stopple retaining collar 1326, and the distal surface of internal sealing support 1226 should preferably be capable of sealing water pressure at 300 kPa in accordance with Part 5.2 of the International Standard ISO 594-2 (1998 ed., Reference No. ISO 594-2: 1998(E)) as published by the International Organization for Standardization, Case postale 56, CH-1211 Geneva 20, Switzerland.

In order to engage the connection between male connector 1002 and access port 1006 thereby allowing fluid flow, adapter ring 1004 is first attached to the luer lock threads of needleless access port 1006. The male connector 1002 and the access port 1006 are then brought into approximate center alignment so that the proximal surface of male connector septum 1220 is in contact with the valve of needleless access port 1006. As discussed in greater detail above, alignment features such as anti-rotation alignment wings 1114 formed on adapter ring 1004 can be aligned with corresponding anti-rotation grooves or slots 1018 formed in male connector housing 1002 in order to ensure proper orientation and to prevent accidental connection of the wrong fluid lines in a multiple connection environment.

As the male connector 1002 and the access port 1006 are pushed together, the surface of male septum 1320 is pressed against the septum (valve) of access port 1006. As the connecting motion continues, the cylindrical center section 1328 of male connector stopple 1310 is compressed axially towards the distal end of the tip 1408 of penetration tube 1402. The tip of penetration tube 1402 is pushed through stopple slit 1324 in male septum 1320 and extends into the interior of access port 1006. Stopple slit 1324 expands radially to accommodate and seal around the increasing diameter of tip 1408 as penetration tube 1402 is pushed further into access port 1006 and male connector stopple 1310 is further compressed. The taper of the cylindrical center section 1328 of male connector stopple 1310 causes the compressed stopple 1310 to push outward and form a seal between the exterior sidewall of cylindrical center section 1328 and the internal wall of the housing of access port 1006. As the connectors are pushed further together, tip 1408 interacts with the valve mechanism of the needleless access port so as to open the fluid connection between the connector and the access port. Once the fluid connection has been established, fluid will be allowed to flow from a fluid source (not shown) through penetration tube 1402, into the needleless access port, and then out into the downstream portion of the fluid-delivery device (not shown). A number of different valve mechanisms are known in the prior art. However, since tip 1408 in conjunction with stopple 1310 will preferably substantially conform to ISO and ANSI standards, the combination of tip and stopple should be able to open fluid connection with any standard needless access port regardless of the valve mechanism employed.

Once the male connector 1002 and the access port 1006 with attached adapter ring 1004 are brought together in the fully engaged position, adapter ring detents 1110 and male connector detents 1218 engage to hold the connectors in place and maintain fluid connection. Although the detents described in this embodiment are essentially latches with lips and opposing faces that catch to hold the detents together, skilled persons will recognize that a number of different types of detents could be used, including but not limited to additional latching mechanisms such as magnetic, adhesive, or hook and loop connections. In a preferred embodiment, engagement of the adapter ring connector detents 1110 and male connector detents 1218 will result in an audible “click” indicating the proper engagement of the two components.

Penetration tube 1402 preferably has a generally cylindrical base 1404, which can be threaded with external luer threads 1406, and a generally cylindrical tip 1408 with fluid transfer opening 1410. Preferably, the shape of the cylindrical tip of penetration tube 1402, when assembled with stopple 1310, approximates ISO and ANSI standards for a male luer tip. External luer threads 1406 on penetration tube base 1402 can be used to connect a fluid transfer line such as a conventional IV line. Optionally, cylindrical base 1404 can be hermetically attached to tubing eliminating the need for luer threads 1406. The interior of base 1404 and tip 1408 are continuous such that liquid flowing into base 1404, for example fluid flowing through an IV line, passes into tip 1408 and exits through fluid transfer opening 1410.

Referring also to FIG. 12B, penetration tube 1402 can be held in place in male connector housing 1222 by tube retainer latches 1215. In a preferred embodiment, when penetration tube 1402 is mounted into male connector housing 1222, tube retaining latches 1215 compress as tube retainer ring 1414 passes, then expand to lock into latch grooves 1420 located on the distal surface of tube retainer ring 1414, thereby holding tube 1402 in its final position. Alternatively, tube 1402 can be held in connector housing 1222 with adhesive bonding or sonic welding. Retaining collar 1326, the flange portion at the distal end of stopple 1310 as discussed above, is compressed between the proximal surface of tube retainer ring 1414 and a sealing bead 1230 on the interior surface of internal sealing support 1226. Preferably, the connection between penetration tube 1402 and male connector housing 1222 is capable of withstanding an axial load of 25N in accordance with International Standard ISO 594-1 (1986 ed., Reference No. ISO 594/1-1986(E)) as published by the International Organization for Standardization, Case postale 56, CH-1211 Geneva 20, Switzerland.

Although the present invention and its advantages have been described in detail, it should be understood that various changes, substitutions and alterations can be made to the embodiments described herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

We claim as follows:

1. A fluid connector for conveying fluid between an I.V. supply line and a standard needleless medical connector, comprising:
a first housing defining an interior and having a proximal and a distal opening, said housing being adapted to fluidly connect to a standard needleless medical connector at its proximal opening, and said housing being configured to be fluidly coupled to an I.V. supply line at its distal opening;
a penetration tube mounted within said first housing, the penetration tube having a proximal and a distal end with a fluid opening on each end, said fluid opening on the distal end being fluidly coupled to the interior of the first housing;
a first stopple formed from an elastic material, said first stopple mounted in the proximal opening of the first housing, said first stopple having a normally closed opening that fluidly seals the proximal fluid opening of the penetration tube, said normally closed opening configured to receive and pass the proximal end of the penetration tube when the housing is connected to a standard needleless medical connector;
said penetration tube and said stopple in combination substantially conforming to ISO or ANSI standards for male conical fittings and suitable for insertion into a standard female conical fitting so that when the first housing is connected to a standard needleless medical connector the proximal opening of the penetration tube passes through the stopple opening and penetrates deeply enough into the needleless medical connector to fluidly connect the fluid opening on the proximal end of the penetration tube with the interior of the needleless medical connector while the stopple forms a fluid seal with the inner diameter of the housing of the needleless medical connector; and
said first housing and said needleless medical connector having an attachment means for securing the said first housing and said needleless medical connector together once they have been connected, wherein said attachment means will release so as to de-couple said first housing and said needleless medical connector when a sufficient pre-selected force is exerted to pull said first housing and said needleless medical connector away from one another.

2. The connector of claim 1, wherein the penetration tube when assembled with the male connector stopple, has a luer taper toward its proximal end of approximately 6%.

3. The connector of claim 1, wherein the first housing is generally cylindrically shaped.

4. The connector of claim 1, wherein the first stopple is substantially cylindrical in shape and having a cylinder axis, the stopple compressing along said cylinder axis when said first housing and said needleless medical connector are connected together thereby unblocking and passing the proximal end of the penetration tube and allowing it to enter the needleless medical connector.

5. The connector of claim 1, wherein said attachment means further comprises an adapter ring threadedly engaged to external threads on the needleless medical connector such that said adapter ring can be connected to said first housing thereby securing the housing and the needleless medical connector together.

6. The connector of claim 5 wherein said adapter ring and said first housing each have at least one set of cooperatively interlocking detents for securing said adapter ring and said first housing together, wherein said at least one set of detents release to de-couple said adapter ring and said first housing when a sufficient pre-selected force is exerted to pull said adapter ring and said first housing away from one another.

7. The connector of claim 6, further comprising a lever release mechanism operably linked to the cooperatively interlocking detents to release said detents in response to a release force exerted by a user.

8. The connector of claim 1, wherein said normally closed opening in the first stopple comprises a slit opening.

9. The connector of claim 1, wherein said attachment means comprises detents formed on said first housing suitable to interlock with standard external luer lock threads.

10. An IV system comprising the fluid connector recited in claim 1.

11. A disconnectable fluid connector, comprising:
a first housing defining an interior and having a proximal and a distal opening;
one or more valves mounted within the first housing, said valve or valves biased in a normally closed position to block any fluid communication between the proximal and distal openings of said first housing;
a penetration tube mounted within said first housing, the penetration tube comprising a conical blunt male cannula substantially conforming to ISO or ANSI standards for male conical fittings, said penetration tube having a proximal and a distal end with a fluid opening on each end, said fluid opening on the distal end being fluidly coupled to the interior of the first housing, said fluid opening on the proximal end being normally blocked by the one or more valves;
attachment means to connect the proximal opening of the first housing to the inlet port of a needleless medical connector conforming to ISO or ANSI standards such that the connection forces said valve or valves into an open position to create a fluid connection pathway allowing fluid communication between the connected first housing and the needleless medical connector; and
said attachment means being detachable when a predetermined distal axial force is exerted to pull the first housing away from the needleless medical connector, thereby operating to disconnect said first housing from said needleless medical connector and to return said valve or valves to a normally closed position.

12. The disconnectable fluid connector of claim 11 wherein said attachment means further comprises an adapter ring threadedly engaged to external threads on female luer conical fitting such that said adapter ring can be connected to the proximal opening of the first housing.

13. The disconnectable fluid connector of claim 11 wherein said attachment means is detachable upon the application of a distal axial force less than said predetermined distal axial force to said housing and said needleless medical connector by way of a mechanical detachment means operated by the application of force other than a distal axial force, thereby operating to disconnect said first housing from said needleless medical connector and to return said valve or valves to a normally closed position.

14. The disconnectable fluid connector of claim 13, wherein once said first housing has been disconnected from said needleless medical connector, the proximal surface of said valve or valves can be disinfected and the first housing and the needleless medical connector sterilely reattached by said attachment means.

15. An IV system comprising the disconnectable fluid connector recited in claim 11.

16. A fluid connector, comprising:
an adapter ring being adapted to connect to the external luer lock threads of a standard needleless medical connector;
a first housing defining an interior and having a proximal and a distal opening, said housing being adapted to connect to said adapter ring at its proximal opening, and said housing suitable to be fluidly coupled to a catheter at its distal opening;
at least one set of cooperatively interlocking break-away detents for securing said adapter ring and said first housing together, said detents releasing to de-couple said adapter ring and said first housing when a sufficient force is exerted to pull said adapter ring and said first housing away from one another;
a penetration tube mounted within said first housing, the penetration tube having distal and proximal ends, said distal end being fluidly coupled to the first housing distal opening, and said penetration tube being suitable for fluidly engaging a standard needleless medical connector;
a first stopple mounted in the proximal opening of the first housing to fluidly seal the proximal opening of the penetration tube when said penetration tube is not fluidly engaged with a needleless medical connector, the first stopple having a normally closed opening for receiving and passing the proximal end of the penetration tube for entry into the inlet of a standard needleless medical connector when the adapter ring has been attached to the external threads of the needleless medical connector and the first housing connected to said adapter ring; and
a lever mechanism operably linked to the at least one set of cooperatively interlocking detents to release said detents and de-couple the first housing from the adapter ring in response to a release force exerted by a user.
